(19) United States
(12) Patent Application Publication
(10) Pub. No.: US 2015/0050337 A1
(43) Pub. Date: Feb. 19, 2015

(54) Extended Release Compositions

(71) Applicants: Subrata Kundu, Verna (IN); Atul Patil, Verna (IN); Nitin Borkar, Verna (IN)

(72) Inventors: Subrata Kundu, Verna (IN); Atul Patil, Verna (IN); Nitin Borkar, Verna (IN)

(73) Assignee: GENERIC PARTNERS PTY. LTD, Hawthorn (AU)

(21) Appl. No.: 13/965,902

(22) Filed: Aug. 13, 2013

(51) Int. Cl. A61K 9/28; A61K 31/194; A61K 31/137

(52) U.S. Cl. CPC A61K 9/28; A61K 31/137; A61K 31/194
USPC 424/465; 514/653; 424/489

(57) ABSTRACT

Provided are controlled release pharmaceutical compositions comprising desvenlafaxine oxalate, one or more release rate controlling polymers, and pharmaceutically acceptable excipients.

### FIELD OF THE INVENTION

The present invention relates, in general, to the field of pharmaceutical compositions and in particular, to pharmaceutical compositions designed for controlled release delivery of desvenlafaxine oxalate.

### BACKGROUND OF THE INVENTION

Desvenlafaxine, chemically described as (±)-1-[2-(dimethylamino)-1-(4-phenol)ethyl]-cyclohexanol, belongs to the serotonin-norepinephrine reuptake inhibitor class of drugs and is an antidepressant having the following chemical structure.

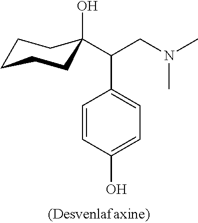

Commonly referred as O-desmethyl venlafaxine, desvenlafaxine is a major metabolite of antidepressant compound venlafaxine, and has been shown to inhibit norepinephrine and serotonin uptake. Worldwide, it is prescribed as antidepressant drug and marketed for the treatment of major depression disorder (MDD) and further being investigated for use in the management of vasomotor symptoms in postmenopausal women.

Desvenlafaxine, reported in the U.S. Pat. No. 4,535,186 was exemplified as a fumarate salt. However, physiochemical and permeability properties of the fumarate salt were found unsuitable for drug development. In drug development, salts are used to alter the physical or chemical properties of a drug substance which can lead to greatly improved stability and bioavailability. For example, succinate salt of desvenlafaxine disclosed in the U.S. Pat. Nos. 6,673,838 and 7,291,347 showed improved solubility and premeability and was found suitable for oral administration. Different salts of desvenlafaxine have been reported in the literature, such as, U.S. Pat. Nos. 4,535,186; 6,673,838; 7,001,920; 8,063,250; 8,269,040; and WO Pub Nos. 2009155488; 2009114685; and 2010088865.

Various controlled release compositions of desvenlafaxine are known and been reported in the literature. For example, U.S. Pat. Nos. 7,470,435 and 7,931,915 teaches controlled release composition containing immediate release pellets and controlled release pellets of desvenlafaxine. The US Pub. No. 20100209489 teaches controlled release formulation containing desvenlafaxine hydrochloride monohydrate and solubility modulator. Another approach describing controlled release formulation of amorphous desvenlafaxine is taught in the US Pub. No. 20120087986.

Despite these advances in the art, there remains a need for reasonably simpler and more practical controlled release compositions of desvenlafaxine.

### SUMMARY OF THE INVENTION

The present invention provides pharmaceutical compositions for orally administrable controlled release delivery of desvenlafaxine oxalate. The pharmaceutical compositions comprise desvenlafaxine oxalate, one or more release rate controlling polymers, and pharmaceutically acceptable excipients.

In a preferred embodiment, the present invention provides an orally administrable pharmaceutical composition comprising desvenlafaxine oxalate, one or more release rate controlling polymer selected from the group consisting of a hydrophilic substance, a hydrophobic substance, and a mixture thereof, and pharmaceutically acceptable excipients.

In a further preferred embodiment, the present invention provides an orally administrable pharmaceutical composition comprising desvenlafaxine oxalate, one or more release rate controlling polymer selected from the group consisting of a hydrophilic substance, a hydrophobic substance, and a mixture thereof, and pharmaceutically acceptable excipients, wherein desvenlafaxine oxalate is in crystalline form and have particle size between 2 to 400 microns.

In a further preferred embodiment, the present invention provides an orally administrable pharmaceutical composition comprises a core containing desvenlafaxine oxalate and one or more pharmaceutically acceptable excipients, and a coating composition containing one or more release rate controlling polymers selected from the group consisting of a hydrophilic substance, a hydrophobic substance, and a mixture thereof.

### BRIEF DESCRIPTION OF DRAWINGS

FIG. 1: Instrinsic dissolution profile of desvenlafaxine from the tablet composition of the present invention.

FIG. 2: Dissolution profile of desvenlafaxine from the tablet composition of the present invention in multimedia.

FIG. 3: Dissolution profile of desvenlafaxine from the tablet composition of the present invention in multimedia.

FIG. 4: Dissolution profile of desvenlafaxine from the tablet composition of the present invention in multimedia.

FIG. 5: Dissolution profile of desvenlafaxine from the tablet composition of the present invention in multimedia.

### DETAILED DESCRIPTION OF THE INVENTION

The present invention provides pharmaceutical compositions comprising desvenlafaxine oxalate in a therapeutically effective amount for orally controlled release administration to a patient, and provides a bioequivalent effect to the reference standard product, i.e. Pristiq®.

The following disclosure describes the pharmaceutical composition which constitutes the invention. The invention is not limited to the specific composition described herein, as such may, of course, vary. It is also to be understood that the terminology used herein is for the purpose of describing particular embodiments only, and is not intended to limit the scope of the present invention.

It must be noted that as used herein and in the appended claims, the singular forms “a”, “and”, and “the” include plural references unless the context clearly dictates otherwise.

Unless defined otherwise, all technical and scientific terms as used herein have the ordinary meaning as understood by those skilled in the art to which this invention belongs.

The term “bioequivalent” or “bioequivalence” refers to, by way of non-limiting example, a drug product or a pharmaceutical composition that, upon administration to a patient, provides calculated 90% confidence interval for AUC and Cₘₐₓ in the range of 80% to 125% of those provided by a reference standard.

The term “composition” and “pharmaceutical composition” are used interchangeably and refer to a controlled release pharmaceutical composition containing desvenlafaxine oxalate for oral administration to a patient.

The term “dissolution” refers to the process by which the active ingredient is dissolved from the pharmaceutical composition in the presence of a solvent, in vitro, or physiological fluid in vivo, e.g. saliva.

The term “excipient” or “pharmaceutically acceptable excipient” means a component of a pharmaceutical composition that is not an active ingredient, such as a filler, diluent, carrier, etc. The excipients that are useful in preparing a pharmaceutical composition are generally safe, non-toxic and neither biologically nor otherwise undesirable, and are acceptable for veterinary use as well as human pharmaceutical use. An “excipient” includes both one and more than one substance.

The term “release rate controlling polymer” as used herein refers to any excipient substance that can alter or modify the drug release time, rate, and extent in any manner from a composition or formulation, such as, for example controlling, sustaining, modifying, prolonging, or delaying the drug release.

In this specification, the term ‘comprises’ and its variants are not intended to exclude the presence of other integers, components or steps.

In a preferred embodiment, the invention provides a pharmaceutical composition for controlled release delivery of desvenlafaxine oxalate for oral administration to a patient. In addition to desvenlafaxine oxalate, the pharmaceutical composition contains one or more release rate controlling polymer, and pharmaceutically acceptable excipients.

In another preferred embodiment, the invention provides a pharmaceutical composition comprising a core matrix comprising therapeutically effective amount of desvenlafaxine oxalate, one or more release rate controlling polymers, and pharmaceutically acceptable excipients.

In still another preferred embodiment, the invention provides an oral controlled release pharmaceutical composition comprising a core containing desvenlafaxine oxalate and a soluble and/or insoluble filler, and a coating composition containing one or more release rate controlling polymer surrounding the core.

In still another preferred embodiment, the invention provides an orally administrable pharmaceutical composition for controlled release delivery of desvenlafaxine oxalate further includes additional pharmaceutically acceptable excipients, including one or more diluents, binders, glidants, lubricants, surfactants, and other commonly used excipients.

In yet another preferred embodiment, the invention provides an orally administrable pharmaceutical composition comprising a core containing desvenlafaxine oxalate and one or more release rate controlling polymers prepared by aqueous/non-aqueous granulation or direct compression or roller compaction.

The release rate controlling polymer used in the present invention includes a hydrophilic substance, a hydrophobic substance, and mixtures thereof. Hydrophilic substance includes, but is not limited to, celluloses such as carboxymethyl cellulose, Xanthun Gum, Na CMC, Na alginate, hydroxypropyl methylcellulose, carboxymethylamide; potassium methacrylate/divinylbenzene copolymers; polyvinylpyrrolidone; gums such as agrose, gum Arabic, gum ghatti, gum karaya, gum tragacanth; hydrophilic colloids such as alginates; other substances such as arbinoglactan, pectin, amylopectin, and the like such as carbopol, PEO, HPMC K100 M CR etc.

Hydrophobic substance includes, but is not limited to, atty acid esters such as glyceryl monostearate, glycerol distearate, glycerol monooleate, acetylated monoglycerides, tristearin, tripalmitin, cetyl esters wax, carnauba wax, Glyceryl palmitostearate, Stearic acid, hydrogenated vegetable oil, hydrogenated castor oil and glyceryl behenate; celluloses such as ethyl cellulose, low substituted hydroxypropyl cellulose (L-HPC), and their derivatives; phthalates such as cellulose acetate phthalate, hydroxypropyl methylcellulose phthalate; cellulose acylate, cellulose diacylate and cellulose triacylate; and cellulose triacetate; mono-, di- and tri-cellulose alkanylates, mono-, di-, and tri-cellulose arylates and mono-, di- and tri-cellulose alkenylates; polymethacrylic acid based polymers and copolymers such as those sold using the trademark EUDRAGIT (RL, RS and NE-30D); copolymers of the above polymers or mixtures of any two or more in various ratios and proportions as required are within the scope of this invention without limitation.

Suitable diluent used in the invention includes microcrystalline cellulose (MCC), silicified MCC (e.g. Prosolv™ HD 90), micro fine cellulose, lactose, starch, pregelatinized starch, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, and the like.

Suitable binder used in the invention includes acacia, guar gum, alginic acid, dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethylcellulose sodium, povidone (e.g. Povidone K−90 D, KOLLIDON®, PLASDONE®) and starch.

The pharmaceutical composition of the present invention comprises desvenlafaxine oxalate and one or more release rate controlling polymers as a blend, with or without pharmaceutically acceptable excipients and may be formulated in the form of tablets, mini-tablets, tablet in tablet, inlay tablets, MUPS (Multi unit particulate system) pellets (extruded or fluidized) or beads or spheres or cores that are either encapsulated or compressed into tablets or minitablets or inlay tablets or capsules encapsulating minitablets or pellets (extruded or fluidized) or both, powders or lyophilized powders and the like.

Inert beads or spheres or cores or seeds or particles or nuclei may comprise water-soluble materials such as sugar spheres, tartaric acid pellets and the like; water-insoluble materials such as microcrystalline cellulose, calcium carbonate, di-calcium phosphate anhydrous, di-calcium phosphate monohydrate, Tri basic calcium phosphate, magnesium carbonate, magnesium oxide and the like. Further drug layering can be done by powder coating or spray coating onto inert particles. The resulting materials may further compressed as tablets. The core tablets or pellets can further be optionally film coated, enteric coated or extended release coated. The coating can be done by techniques known to one skilled in the art such as spray coating, dip coating, fluidized bed coating and the like.

The compositions for tabletting may further include additional pharmaceutically acceptable excipients, including one or more of glidants, lubricants, surfactants and other commonly used excipients.

The process for manufacturing the pharmaceutical composition of present invention is not limited to the processes described in the application and the composition can be prepared by using any of the processes known to one skilled in the art. The active ingredient can be granulated by wet granulation or dry granulation with or without excipients. The granules of active(s) are prepared by sifting the actives and excipients through the desired mesh size sieve and then are mixed using a rapid mixer granulator or planetary mixer or mass mixer or ribbon mixer or fluid bed processor or any other suitable device. The blend can be granulated, such as by adding a solution of a binder whether aqueous or alcoholic or hydro-alcoholic in a low or high shear mixer, fluidized bed granulator and the like. The granulate can be dried using a tray drier or fluid bed drier or rotary cone vacuum drier and the like. The sizing of the granules can be done using an oscillating granulator or comminuting mill or any other conventional equipment equipped with a suitable screen. Alternatively, granules can be prepared by extrusion and spheronization, or roller compaction.

Alternatively the manufacture of granules may be done by direct compression by mixing the directly compressible excipients with active(s). The blend so obtained can either be compressed using a suitable device, such as a multi-station rotary machine to form compressed slugs or by roller compaction to form slugs, which are passed through a multimill, fluid energy mill, ball mill, colloid mill, roller mill, hammer mill, or the like, equipped with a suitable screen. The milled slugs are then mixed with lubricants if required.

The pharmaceutical composition of the present invention can be in the form of inlay tablet. An inlay tablet is a compressed solid oral dosage form which has a small tablet placed within a large tablet, such that three sides of a small tablet are within a large tablet and only one surface of the small tablet is exposed. Exposure of a surface of the smaller tablet is achieved by contact of that surface with the surface of a compression die, and placing the granulate or powder mixture over the smaller tablet before the final compression step. Desvenlafaxine oxalate can be present as a small tablet placed within a large tablet that is containing one or more release rate controlling polymers. The tablets can further be optionally film coated or enteric coated or coated with control release polymers. The coating can be done by techniques known to one skilled in the art such as spray coating, dip coating, fluidized bed coating and the like.

Suitable solvent system used in the granulation or coating include purified water, ethanol, isopropyl alcohol, acetone, methylene chloride and the like.

Unless otherwise specified this invention is directed towards achieving the ascending type release comparable to Pristiq® extended release tablets using release rate controlling polymers such that it follows first order and Higuchi release mechanism.

Ascending type release as defined here, the release of NMT 40% in 2 hrs and NMT 55% in 4 hrs NLT 65% in 8 hrs, NLT 75 in 12 hrs & NLT 80 in 24 hrs and when the values of the test product are compared with PRISTQ® shows F2 (similarity factor) greater than 50.

Desvenlafaxine oxalate used in the pharmaceutical composition of the present invention is in crystalline form and the particle size is between 2 to 400 microns. Preferably, more than 90% of the particles have a particle size between 2 to 200 microns and more preferably in 100 micron.

The following examples will further illustrate certain aspects of the invention in greater detail and are not intended to limit the scope of the invention.

### Example 1

### Desvenlafaxine Oxalate Tablet Composition

Desvenlafaxine oxalate and other excipients except lubricants were sifted through ASTM #30 and used directly for direct compression with extra granular lubricants. The granular mass is dried. Lubricants sifted through ASTM #40 were blended with dried granules and compressed with hydraulic press for performing intrinsic dissolution test.

TABLE 1

| | B1 | B2 | B3 | B4 | B5 |
|---|---|---|---|---|---|
| Ingredients | % w/w | % w/w | % w/w | % w/w | % w/w |
| Desvenlafaxine oxalate | 19.70 | 19.70 | 19.70 | 19.70 | 19.70 |
| Hypromellose 2208 | 50 | 40 | — | — | 60 |
| Hypromellose 2208 | — | — | 40 | — | — |
| Polyacrylic acid | — | — | — | 40 | — |
| Microcrystalline | 25 | 35 | 35 | 35 | 15 |
| cellulose | | | | | |
| Talc | 2.65 | 2.65 | 2.65 | 2.65 | 2.65 |
| Magnesium stearate | 2.65 | 2.65 | 2.65 | 2.65 | 2.65 |
| Total (% w/w) | 100 | 100 | 100 | 100 | 100 |

### Example 2

### Release Profile

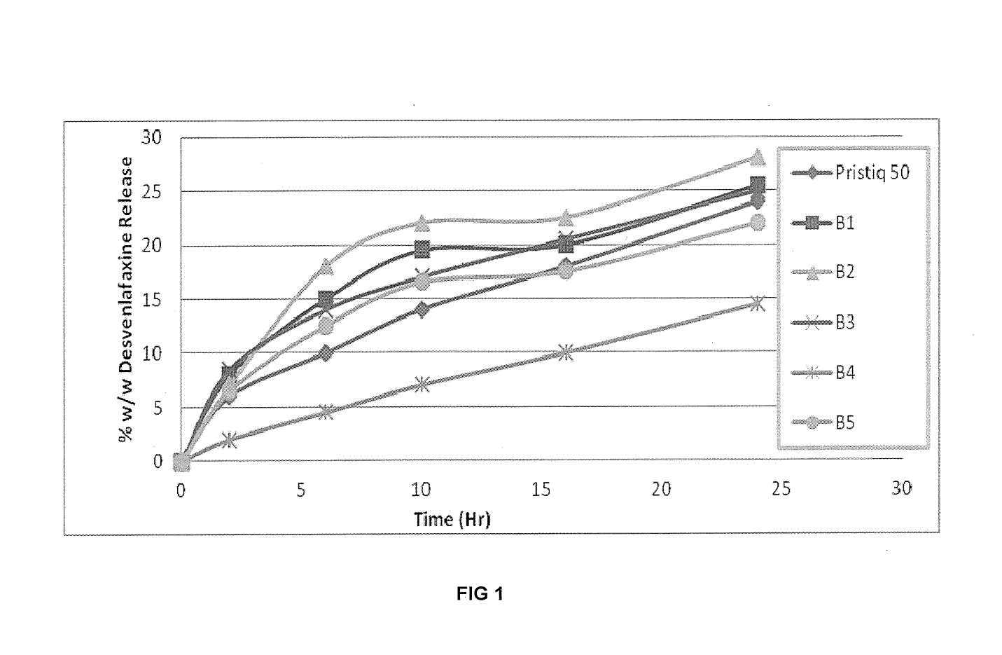

Intrinsic dissolution of the composition of Table 1 was performed in pH 6.8 phosphate buffer with 500 ml media volume at 25 rpm speed and compared with the reference product Pristiq® as shown in FIG. 1.

TABLE 2

| Time (Hr) | Pristiq 50 | B1 | B2 | B3 | B4 | B5 |
|---|---|---|---|---|---|---|
| 2 | 6 | 8 | 7 | 9 | 2 | 7 |
| 6 | 10 | 15 | 18 | 14 | 5 | 13 |
| 10 | 14 | 20 | 22 | 17 | 7 | 17 |
| 16 | 18 | 20 | 23 | 21 | 10 | 18 |
| 24 | 24 | 26 | 28 | 25 | 15 | 22 |

### Example 3

### Desvenlafaxine Oxalate Tablet Composition

Desvenlafaxine oxalate and other excipients except lubricants were sifted through ASTM #30. The mixture is dried. Lubricants sifted through ASTM #40 were blended with dried mix for 5 minutes and compressed into tablet and performed dissolution test.

TABLE 3

| | B10 | B13 | B14 |
|---|---|---|---|
| Ingredients | % w/w | % w/w | % w/w |
| Desvenlafaxine oxalate | 19.70 | 19.70 | 19.70 |
| Hypromellose 2208 | 40 | 40 | — |
| Hypromellose 2208 | 20 | — | — |
| HPMC 2910 | — | 19.70 | 19.70 |
| Microcrystalline cellulose | 17.30 | 17.60 | 37.58 |
| Talc | 2 | 2 | 2 |
| Magnesium stearate | 1 | 1 | 1 |
| Total (% w/w) | 100 | 100 | 100 |

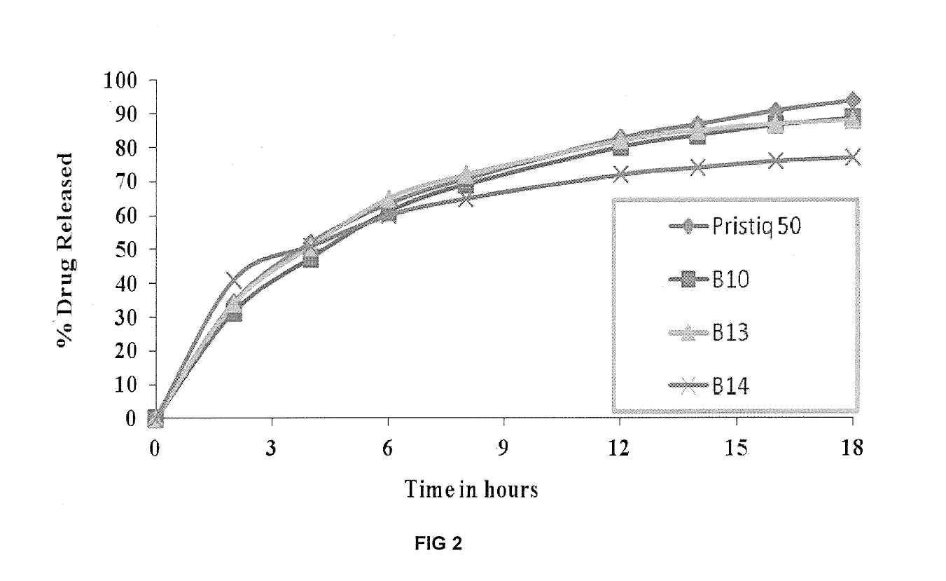

Multimedia dissolution was performed in 0.1N HCl, pH 4.5 acetate and pH 6.8 phosphate buffers with 500 ml media volume at 75 rpm spied with USP type I basket and compared with the reference product Pristiq® as shown in FIG. 2.

### Example 4

### Tablet Containing Hydrophilic Substance as Release Rate Controlling Polymer

Desvenlafaxine oxalate and other excipients except lubricants were sifted through ASTM #30. The mixture is dried. Lubricants sifted through ASTM #40 were blended with dried mix for 5 minutes and compressed into tablet and performed dissolution test.

TABLE 4

| | C1 | C2 | C3 |
|---|---|---|---|
| Ingredients | % w/w | % w/w | % w/w |
| Desvenlafaxine oxalate | 21.92 | 21.84 | 24.85 |
| Hypromellose 2208 | 40 | — | 45.33 |
| Hypromellose 2208 | 20 | 20 | — |
| HPMC 2910 | — | — | 22.66 |
| Microcrystalline cellulose | 15.08 | 15.16 | 4.16 |
| Talc | 2 | 2 | 2 |
| Magnesium stearate | 1 | 1 | 1 |
| Total (% w/w) | 100 | 100 | 100 |

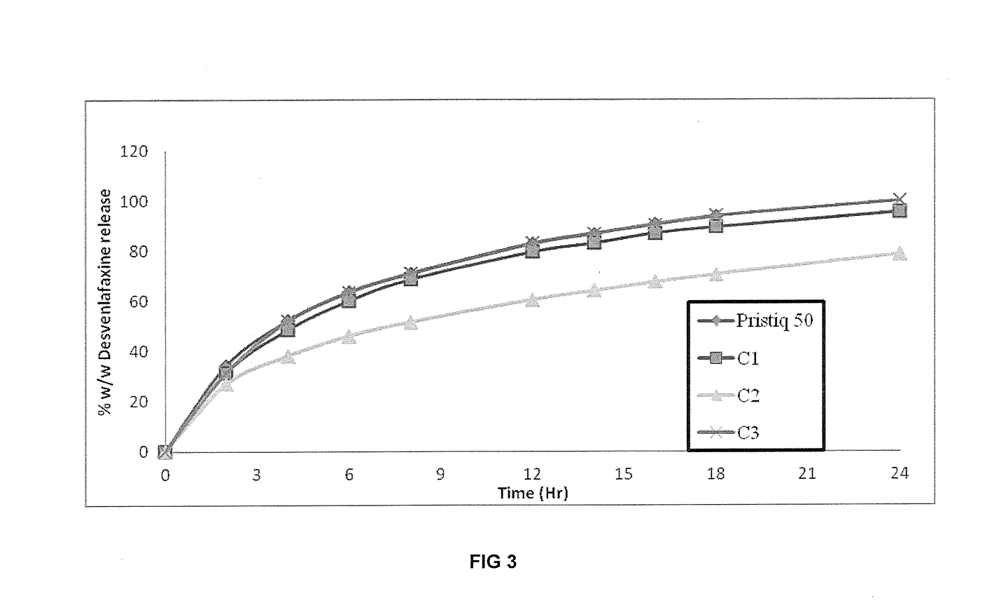

Multimedia dissolution was performed in 0.1N HCl, pH 4.5 acetate and pH 6.8 phosphate buffers with 500 ml media volume at 75 rpm speed with USP type I basket and compared with the reference product Pristiq® as shown in FIG. 3.

### Example 5

### Tablet Containing Hydrophobic Substance as Release Rate Controlling Polymer

TABLE 5

| | C4 | C5 |
|---|---|---|
| Ingredients | % w/w | % w/w |
| Desvenlafaxine oxalate | 21.92 | 21.92 |
| Sterotex | 40 | — |
| Lactose | 20 | 20 |
| Compretrol | — | 40 |
| Microcrystalline cellulose | 15.08 | 15.08 |
| Talc | 2 | 2 |
| Magnesium stearate | 1 | 1 |
| Total | 340 | 340 |

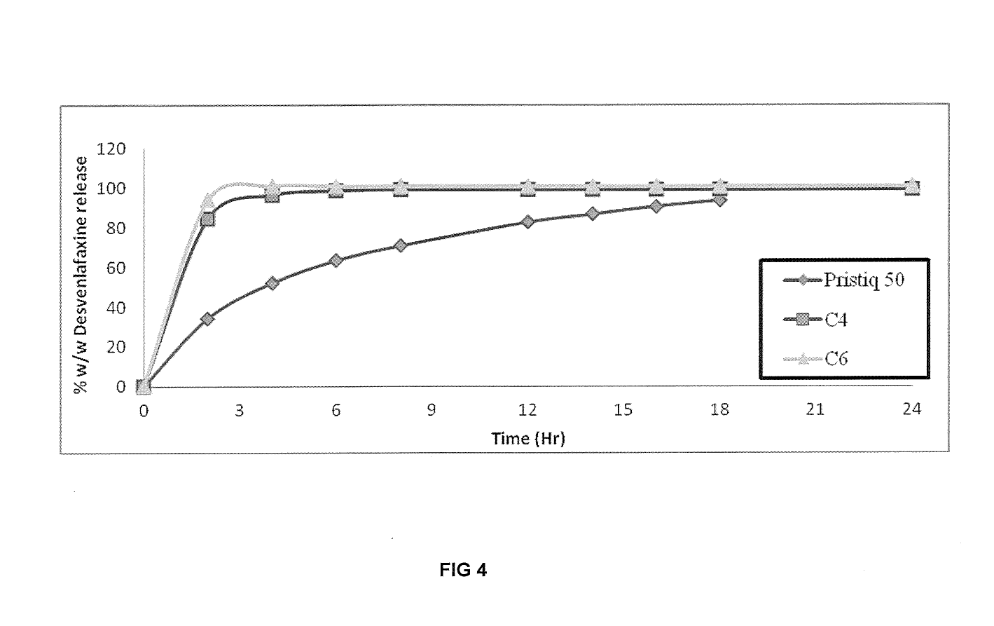

Dissolution profile of both the reference standard product Pristiq® and the composition having hydrophobic substance as release rate controlling agent were compared and shown in FIG. 4.

### Example 6

### Tablet Containing Release Rate Controlling Polymer Coating

Desvenlafaxine oxalate and other excipients were sifted through ASTM #30. The mixture is dried. Lubricants sifted through ASTM #40 were blended with dried mix for 5 minutes and compressed into tablet and coated with combination of ethyl cellulose and hydroxyl propyl methylcellulose polymer and samples were withdrawn at 5%, 7.5%, 10%, 15% and 20% weight gain.

TABLE 6

| | |
|---|---|
| Ingredient | % w/w |

Core composition

| | |
|---|---|
| Desvenlafaxine oxalate | 24.85 |
| Lactose | 17.08 |
| Microcrystalline cellulose | 49.00 |
| Povidone | 5.66 |
| Talc | 2.26 |
| Magnesium stearate | 1.13 |
| Total | 100 |

ER coat composition

| | |
|---|---|
| Ethyl cellulose | 1.70 |
| HPMC | 0.30 |
| Tri ethyl citrate | 0.30 |
| Methylene chloride | q.s. |
| Isopropyl alcohol | q.s |

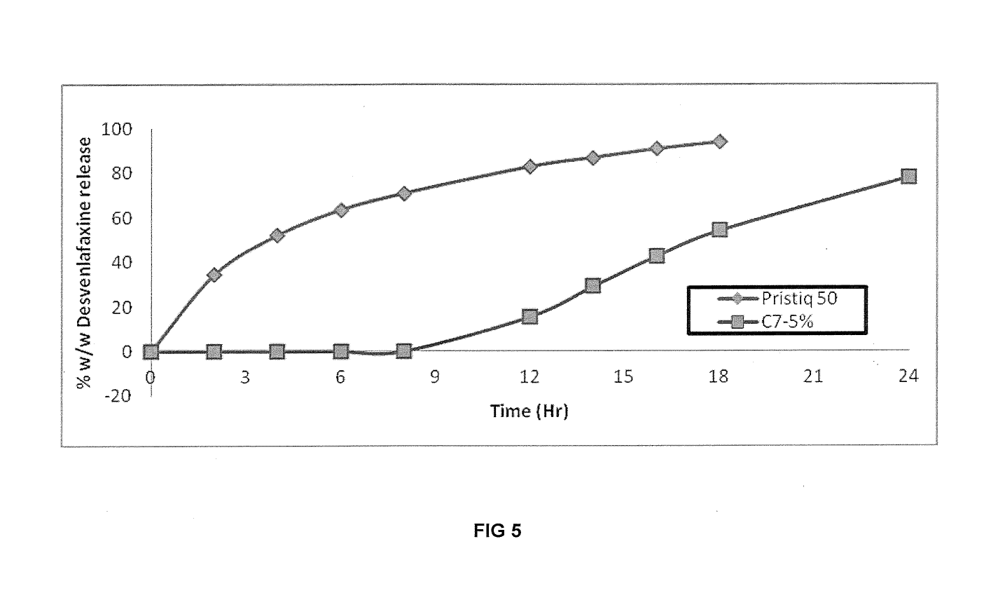

Multimedia dissolution was performed in 0.1N HCl, pH 4.5 acetate and pH 6.8 phosphate buffers with 500 ml media volume at 75 rpm speed with USP type I basket and compared with the reference product Pristiq® as shown in FIG. 5.

It is a scope of this invention is to provide a pharmaceutical composition containing desvenlafaxine oxalate and one or more release rate controlling polymers to modulate the release of desvenlafaxine from core in such a way that when compared to reference product Pristiq® similarity factor F2 is always greater than 50 for dissolution data or values generated in any suitable dissolution method. The product developed essentially follows first order or Higuchi release mechanism, thus indicating release by diffusion principle.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In this specification, references to prior art are not intended to acknowledge or suggest that such prior art is part of the common general knowledge in Australia or that a person skilled in the relevant art could be reasonably expected to have ascertained, understood and regarded it as relevant.

1. An orally administrable pharmaceutical composition comprising desvenlafaxine oxalate, one or more release rate controlling polymer selected from the group consisting of a hydrophilic substance, a hydrophobic substance, and a mixture thereof, and pharmaceutically acceptable excipients.

2. The composition as claimed in claim 1, wherein the composition further comprises a binder, a lubricant and a diluent.

3. The composition as claimed in claim 1, wherein the desvenlafaxine oxalate is in crystalline form.

4. The composition as claimed in claim 1, wherein the desvenlafaxine oxalate have particle size between 2 to 400 microns.

5. The composition as claimed in claim 1, wherein the desvenlafaxine oxalate have particle size between 2 to 200 microns.

6. The composition as claimed in claim 1, wherein the desvenlafaxine oxalate have particle size of 100 microns.

7. An orally administrable pharmaceutical composition comprising desvenlafaxine oxalate, one or more release rate controlling polymer selected from the group consisting of a hydrophilic substance, a hydrophobic substance, and a mixture thereof, and pharmaceutically acceptable excipients, wherein desvenlafaxine oxalate is in crystalline form and have particle size between 2 to 400 microns.

8. An orally administrable pharmaceutical composition comprises a core containing desvenlafaxine oxalate and one or more pharmaceutically acceptable excipients, and a coating composition containing one or more release rate controlling polymers selected from the group consisting of a hydrophilic substance, a hydrophobic substance, and a mixture thereof.

9. The composition as claimed in claim 8, wherein the desvenlafaxine oxalate is in crystalline form and has a particle size between 2 to 400 microns.

10. The composition as claimed in claim 1, wherein the composition is controlled release tablet.

11. (canceled)

12. The composition as claimed in claim 7, wherein the composition is controlled release tablet.

13. The composition as claimed in claim 8, wherein the composition is controlled release tablet.
